(19) United States
(12) Patent Application Publication
(10) Pub. No.: US 2005/0203470 A1
(43) Pub. Date: Sep. 15, 2005

(54) Radiographically detectable object assemblies and surgical articles comprising same

(76) Inventor: Marlin Daniel Ballard, Austin, TX (US)

Correspondence Address: DAVID ODELL SIMMONS, 7637 PARKVIEW CIRCLE, AUSTIN, TX, 78731, US

(21) Appl. No.: 11/060,326

(22) Filed: Feb. 17, 2005

Related U.S. Application Data
(63) Continuation-in-part of application No. 10/124,534, filed on Apr. 17, 2002, now Pat. No. 6,777,623.

(51) Int. Cl. A61F 13/15

(52) U.S. Cl. 604/362

(57) ABSTRACT

Apparatuses and methods are provided that employs a “radiopaque” object to count and account for surgical sponges in an operating room. A radiopaque object is attached to surgical sponges so that a scanning device can detect and count a collection of the sponges following use in a surgical procedure. Such apparatuses and methods enable surgical team personnel to insure that no surgical sponge is left in a patient without performing the messy and time-consuming job of individually counting sponges as they are entered and disposed of from the surgical site. In one embodiment, the radiopaque object is provided in the form of a detectable object structure. The detectable object structure includes an object attachment structure capable of being attached to the surgical sponge. The radiopaque object is fixedly engaged by the object attachment structure for limiting movement of the radiopaque object relative to the sponge.

### RELATED APPLICATION

This patent application is a continuation-in-part of the patent application having Ser. No. 10/124,534, filed on Apr. 17, 2002, entitled “System and Method of Tracking Surgical Sponges” and having a common applicant herewith.

### FIELD OF THE DISCLOSURE

The invention relates generally to apparatuses and methods for tracking surgical supplies and, more specifically, to facilitating presence and/or counting of articles capable of absorbing fluids within a body and packing internal bodily structures.

### BACKGROUND

During surgical procedures, articles such as absorbent sponges are employed to soak up blood and other fluids in and around an incision site. In a study entitled “The Retained Surgical Sponge” (Kaiser, et al., The Retained Surgical Sponge, Annals of Surgery, vol. 224, No. 1, pp. 79-84), surgical sponges were found to have been left inside a patient following surgery in 67 of 9729 (0.7%) medical malpractice insurance claims reviewed. In those 67 cases, the mistake was attributed to an incorrect sponge count in seventy-six percent (76%) of the cases studied, and attributed to the fact that no count was performed in ten percent (10%) of the cases studied. Typically, a sponge left inside a patient is presumed to indicate that substandard and negligent care has taken place. Clearly, it is in both a patient's and the health care providers' best interest to account for every surgical sponge used in any particular surgical procedure.

As explained in U.S. Pat. No. 5,923,001 entitled Automatic Surgical Sponge Counter and Blood Loss Determination System, sponge counts are an essential step in operating room procedure. Sponge counts are a difficult procedure for a number of reasons. For example, the handling of soiled sponges carries the risk of transmission of blood borne diseases such as hepatitis B virus (HBV) and human immunodeficiency virus (HIV). Therefore, used sponges are handled with gloves and/or instruments and the handling is kept to a minimum. Another difficulty is that the counting process is typically tedious, time-consuming and frustrating.

Sponge counts are typically performed multiple times during a surgical procedure, both at the beginning and throughout the procedure as sponges are added, before closure of a deep incision or body cavity, and during personnel breaks and shift changes. Thus, within all the activity of an operating room, maintaining an accurate sponge is difficult, as evidenced by the error rate mentioned in the Keiter article, quoted above.

There do exist products to make the procedure both simpler and more reliable. For example, various systems facilitate the hand-counting of surgical sponges by arranging the sponges into visually inspectible groups or arrangements (see U.S. Pat. No. 3,948,390, No. 4,364,490, No. 4,784,267, No. 4,832,198, No. 4,925,048 and No. 5,658,077). These systems are problematic because surgeons and anesthesiologists often determine blood loss by means of visual inspection or a manual weighing of soiled sponges and so soiled sponges are typically kept in one area of an operating room during a surgical procedure, thus creating the possibility that groupings are co-mingled or counted twice. In addition, operating room workers are often too rushed, fatigued and/or distracted to accurately count a large number of soiled sponges lumped together in one or more groups. This method also depends upon the accuracy of an initial count and, if the number of sponges in the original package is mislabeled by the manufacturer, then a missing sponge may be missed during a final count.

A second solution to the surgical sponge tracking problem is the inclusion of a radiopaque thread in the sponges. A radiopaque thread can be identified and located if a sponge is accidentally left inside a patient. Thus, if a patient develops a problem such as an abscess, a bowel obstruction, or internal pain at any time following an operation, a sponge that has been left in the body can be detected by x-ray. Companies that market sponges with radiopaque threads include Johnson & Johnson, Inc. of New Brunswick, N.J., Medline Industries of Mundelein, Ill. and the Kendall Company of Mansfield, Mass.

A third solution to the sponge problem is the inclusion of a radio frequency identification (RFID) tag in each sponge (see U.S. Pat. No. 5,923,001). The RFID tag enables a patient to be scanned to detect the presence of a sponge within a body cavity, but RFID tags may cost several times what a typical surgical sponge costs and are also bulky, impairing the usefulness of the sponge.

Another solution to the sponge problem is a device that counts sponges as they are dropped, one-by-one, into an opening, or “entry gate,” of the device (see U.S. Pat. No. 5,629,498). This solution is restricted by the accuracy of the original count and the precision of operating room assistants as they separate sponges from one another and drop them into the entry gate, one-by-one.

A final, exemplary solution involves attaching a magnetic resonance device, or marker tag, to each sponge, which are then scanned by appropriate equipment (see U.S. Pat. No. 5,057,095 and U.S. Pat. No. 5,664,582). The problem with this solution is that both the marker tags and the scanning equipment are expensive and do not necessarily work well in an operating room environment. As acknowledged in the '582 patent, the scanner must be essentially parallel to the marker tag inside a wadded up sponge. If the marker tag is bent or folded, a signal from the tag may be difficult to identify. In addition, the scanning equipment may give false counts if the operating room contains objects, other than the marker, that also generate or respond to magnetic energy.

Many other problems and disadvantages of the prior art will become apparent to one skilled in the art after comparing such prior art with the present invention as described herein.

### SUMMARY OF THE INVENTION

Embodiments of apparatuses and methods in accordance with the inventive disclosures made herein employ one or more “radiopaque” objects to facilitate counting and/or accounting for articles capable of absorbing fluids within a body and/or packing internal body structures in an operating room. Such articles are generally referred to herein as surgical sponges. The term “radiopaque” refers to an object that is detectable by a scanning device using an x-ray or other penetrating wave or particle such as neutron beams or gamma rays, and infrared, near-infrared, laser, electromagnetic or radio waves. Within the context of the claimed subject matter, a “surgical sponge” is any device or material used in human or animal surgery for the purpose of absorbing blood or other fluids, or for packing, packing off, containing, or isolating (i.e., packing) internal bodily structures within a surgical field.

A radiopaque object is attached to each surgical sponge so that a scanning device can detect and count a large number of the sponges within a container designed to eliminate the need for contact by humans with the sponges. In this manner, a surgical team can insure that no surgical sponge is left in a patient without performing the messy and time-consuming job of individually counting sponges as they are entered and removed from the surgical site.

The claimed subject matter includes specially designed surgical sponges for use with the scanning device. Also included in the claimed subject matter is the use of radiopaque objects of differing configurations (e.g., sizes and/or types) attached to (e.g., embedded in) surgical sponges of differing configurations (e.g., sizes and/or types). For example, a large sponge may contain a large object and a small sponge may contain a small object so that the scanning device can distinguish and count multiple sizes and types of sponges. In one embodiment of the invention, the scanning device also weighs discarded surgical sponges so that a calculation can be made of the sponges' retained fluids, i.e. patient fluid loss.

Various embodiments of detectable object assemblies are disclosed herein. Such detectable object assemblies include a radiopaque object in accordance with the inventive disclosures made herein and means for facilitating attachment of the radiopaque object to material configured for absorbing fluids within a body and/or packing bodily structures. The usefulness of such detectable object assemblies is that they permit small, discrete radiopaque objects to be reliably, efficiently and consistently attached to such material. Examples of such material configurations include single or multiple layers of material comprised by woven material, non-woven material, foam material and the like. In one example, such material is provided in the form of a surgical sponge.

As will be appreciated in view of the embodiments of detectable object assemblies presented herein, the detectable object assemblies may be provided at a particular point of attachment in an article manufacturing process and may be provided in any number of different formats. Examples of such formats include, but are not limited to, a roll of attached assemblies, a magazine of discrete assemblies, a magazine of attached assemblies, a magazine of continuous stock (e.g., extruded stock) from which individual detectable object assemblies are segmented and the like.

In one embodiment of the present invention, a detectable surgical article comprises material configured for at least one of absorbing fluids within a body and packing bodily structures, a radiopaque object and means configured for attaching the radiopaque object to the material. The radiopaque object is configured for producing predictable profiles when scanned while orientated in different positions. The means configured for attaching the radiopaque object to the material is attached to the material and the radiopaque object is fixedly engaged by the means configured for attaching the radiopaque object to the material.

In another embodiment of the present invention, a detectable surgical article comprises material, an object attachment structure attached to the material and a radiopaque object configured for producing predictable profiles when scanned while orientated in different positions. The material is configured for at least one of absorbing fluids within a body and packing bodily structures. The radiopaque object is fixedly engaged by the object attachment structure.

In another embodiment of the present invention, a detectable object structure comprises a radiopaque object configured for producing predictable profiles when scanned while orientated in different positions and an object attachment structure including an object-receiving portion having the radiopaque object at least partially disposed therein.

Other systems, methods, features and advantages of the invention will be or will become apparent to one with skill in the art upon examination of the following figures, which are not necessarily drawn to scale, and detailed description. It is intended that all such additional systems, methods, features and advantages be included within this description, be within the scope of the invention, and be protected by the accompanying claims.

### BRIEF DESCRIPTION OF THE FIGURES

While various embodiments of the invention have been described, it will be apparent to those of ordinary skill in the art that many more embodiments and implementations are possible that are within the scope of this invention.

FIG. 1 is an exemplary surgical supply tracking system (SSTS) employing the techniques of the claimed subject matter.

FIG. 2 is an exemplary PC-based SSTS employing the techniques of the claimed subject matter.

FIG. 3 is an illustration of a surgical sponge in relation to a radiopaque object according to the claimed subject matter.

FIG. 4 is an illustration of an exemplary surgical sponge in which the radiopaque object is woven or glued into the surgical sponge.

FIG. 5 is an illustration of an exemplary surgical sponge in which the radiopaque object is affixed to the surgical sponge by means of a fixture patch.

FIG. 6 is an illustration of an exemplary surgical sponge in which the radiopaque object is affixed to the surgical sponge by means of a fixture thread.

FIG. 7 is an illustration of an exemplary surgical sponge in which the radiopaque object is affixed to the surgical sponge by means of both a fixture patch and a fixture thread.

FIG. 8 is a flowchart that illustrates the processing performed by the SSTS.

FIG. 9 is a flow chart that illustrates a method configured for enabling system-assisted counting and, optionally, system-performed counting of surgical sponges, wherein the method and surgical sponges employ techniques of the claimed subject matter.

FIGS. 10-12 depict an embodiment of a detectable object structure configured for being attached to a surgical article via a plurality of spaced-apart engagement members.

FIGS. 13-15 depict an embodiment of a detectable object structure configured for being attached to a surgical article via a bonding element.

FIGS. 16-18 depict an embodiment of a detectable object structure configured for being attached to a surgical article via a single engagement member.

FIG. 19 depicts an embodiment of an extruded detectable object structure.

FIG. 20 depicts an embodiment of a detectable surgical article including a pair of engaged bodies and having a surgical article disposed between the engaged bodies.

FIG. 21 depicts an embodiment of a segment of continuously formed detectable object assemblies.

FIGS. 22 and 23 depict an embodiment of a flexible detectable object structure.

FIG. 24 depicts an embodiment of a one-piece detectable object structure attached to a surgical article.

### DETAILED DESCRIPTION OF THE FIGURES

Although described with particular reference to a system for tracking surgical supplies within an operating room, the surgical supply tracking system (SSTS) of the disclosed subject matter can be implemented in any system in which it is desirable to count and/or track objects with a minimum of handling and a very high degree of accuracy.

Selected portions of the SSTS can be implemented in software, hardware, or a combination of hardware and software. Hardware portions of the invention can be implemented using specialized hardware logic. Software portions can be stored in a memory and executed by a suitable computing system such as a microprocessor or a personal computer (PC). Furthermore, software of the SSTS, which comprises an ordered listing of executable instructions for implementing logical functions, can be embodied in any computer-readable medium for use by or in connection with the computing system.

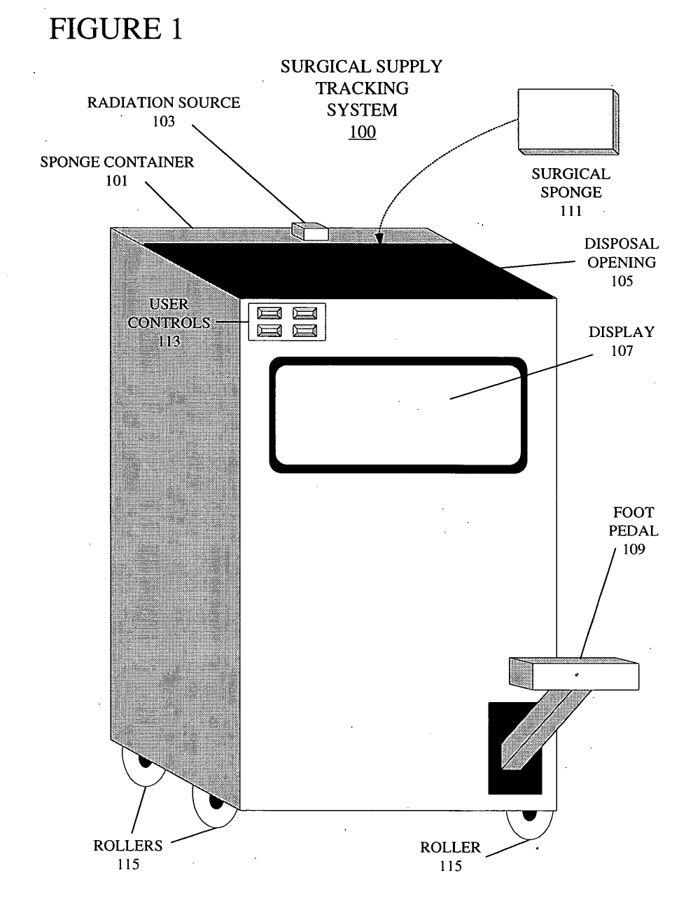

Turning now to the figures, FIG. 1 illustrates an exemplary SSTS 100 for use in an operating room. A sponge container 101 includes a disposal opening 105 through which surgical sponges, such as a surgical sponge 111, are placed after use. For the purposes of this disclosure, a “surgical sponge” is any device or material used in either human or animal surgery for the purpose of absorbing blood or fluids, or for packing, packing off, containing, or isolating internal bodily structures within a surgical field. The sponge container 101 includes rollers 115 to facilitate its movement within and outside the operating room. By pressing a foot pedal 109, a user of the SSTS 100 opens a door (not shown) in the disposal opening 105 so that the used surgical sponge 111 can be placed into the sponge container 101. In addition, the pressing of the foot pedal 109 causes hardware and/or software logic (not shown) in the SSTS 100 to activate a radiation source 103. The hardware and/or software logic, with input from a sensor (not shown), then calculates the number of sponges in the sponge container 101. Once the hardware and/or software logic has calculated the number of sponges in the sponge container 101, this number is displayed on a display 107. It should be apparent to those with skill in the electronic arts that the hardware and/or software logic of the SSTS 100 can be implemented in a number of ways, including, but not limited to, specialized circuits incorporating both hardware and software components.

The sponge container 101 also includes a clear plastic covering (not shown) such as a plastic bag or a form-fitted covering that fits into the disposal opening 105, thus containing the surgical sponges 111, and drapes over the outside of the container 101 in order to keep fluids from the surgical sponges 111 from contaminating the surface of the container 101 and its components. In addition to the number of sponges in the container 101, the display 107 may also display a calculation of the weight of the contained sponges so that operating room personnel can determine patient fluid loss. A set of user controls 113 is employed to turn the SSTS 100 on or off, initiate the display 107 and calibrate the sensors. In alternative embodiments of the SSTS 100, the calculation of the sponges in the container 101 and the display of this number may also be initiated by the user controls 113 rather than, or in addition to, the depression of the foot pedal 109.

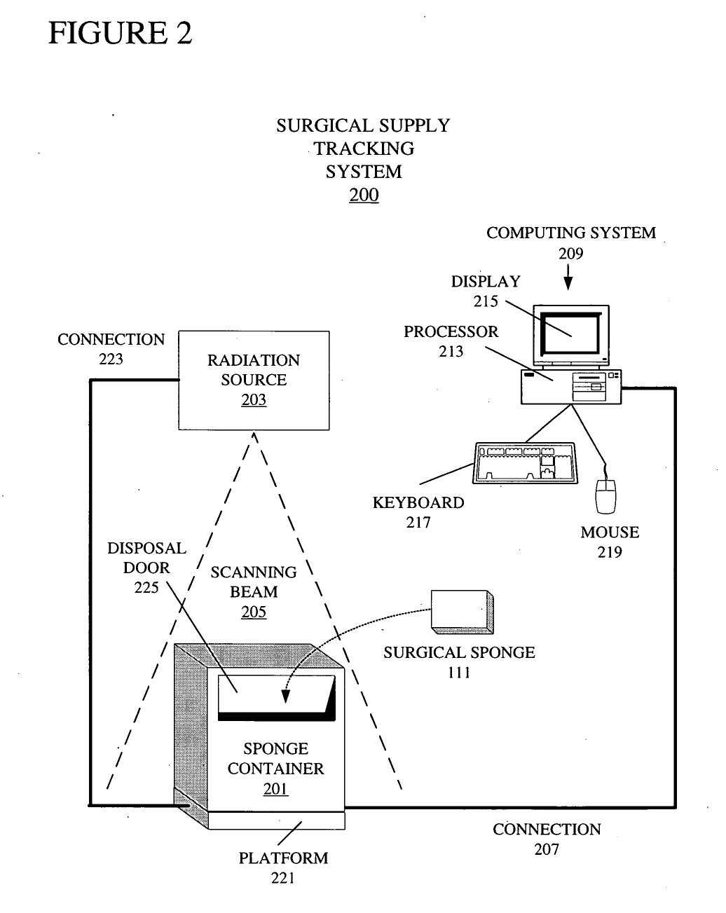

FIG. 2 illustrates an exemplary PC-based SSTS 200 employing the techniques of the claimed subject matter. The SSTS 200 includes a sponge container 201 in which surgical sponges, such as the surgical sponge 111 (FIG. 1), can be disposed following the sponge's 111 use in a surgical procedure. The container 201 is positioned on a platform 221 that is connected via a connection 223 to a radiation source 203, which is similar to the radiation source 103 (FIG. 1). The platform may also include a weight sensor (not shown) for measuring the weight of the container 201 and its contents. The platform 221 is also connected via a connection 207 to a computing system 209. The connections 223 and 207 may be hard-wired, wireless or network connections. In this example, the computing system 209 includes a processor 213, a display 215, a keyboard 217 and a mouse 219. The exact configuration of the computing system 209 is not critical to the spirit of the invention. For example, all or portions of the computing system 209 may be incorporated into the platform 221 in order to provide a compact and integrated system with fewer discrete pieces than the illustrated system 200.

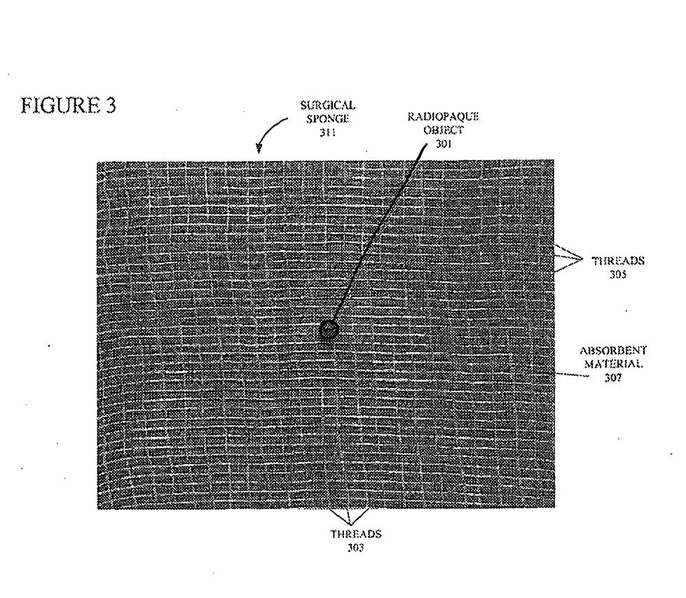

The radiation source 203 emits a scanning beam 205 that enables detectors (not shown) in the platform to detect a small radiopaque object 301 (see FIGS. 3-7) in each sponge 111 in the container 201. The term “radiopaque” means the object 301 is able to obscure or block some type of scanning beam 205 such as x-ray or other penetrating wave or particle such as neutron beams, gamma rays, infrared, near-infrared, laser, electromagnetic waves or radio waves. The specific type of scanning beam 205 is not critical to the spirit of the inventions other than that the detectors in the platform 201 must be able to detect the scanning beam 205 with sufficient resolution to count each radiopaque object 301 in each sponge 111 in the container 101. As with the computing system 209, the radiation source 203 and the platform may be integrated into a single device, in which case the SSTS 200 would look more like the SSTS 100 (FIG. 1).

FIG. 3 is an illustration of a surgical sponge 311 (FIG. 1) in relation to a radiopaque object 301. The surgical sponge 311 is one embodiment of the surgical sponge 111 (FIGS. 1 and 2). The surgical sponge 311 is comprised of an absorbent material 307 contained within vertical threads 303 and horizontal threads 305 (i.e., a woven material). Such woven material may be single layer or multiple layers. Other examples of suitable surgical sponges include foam sponges or other sponges made of non-woven, non-knitted or non-fabric material. The surgical sponge 311, except for the radiopaque object 301, should be familiar to those with experience with surgery and the equipment employed in surgery. Although not necessarily drawn to scale, the radiopaque object 301 is small in relation to the surgical sponge 311. Typically, the radiopaque object 301 is less than one (1) centimeter wide in any direction. Although, the radiopaque object 301, illustrated in FIG. 3, is a metal sphere there can be different types of radiopaque objects; i.e., many different shapes and materials can be employed. For example, the radiopaque object 301 may be cylindrical, cubic, rectangular, triangular or some other polygon, either regularly or irregularly shaped. The radiopaque object 301 may also be some other shape such as a hexagonal nut, either with or without a hole in the middle. The objective of the shapes of a radiopaque objects in accordance with the inventive disclosures made herein is that they produces predictable profiles when scanned while orientated in different positions. In this manner, such predictable profiles enable individual radiopaque objects within an image to be identified and, thereby, counted.

Different configurations (e.g., types or sizes) of radiopaque objects can be used to indicate different configurations (e.g., types or sizes) of surgical sponges. In addition, the radiopaque object may be something other than metal. For example, the object 301 may be barium sulfate encased in a non-water-soluble material such as plastic, latex, rubber, silicone or silastic, or even encased in a tightly woven fabric.

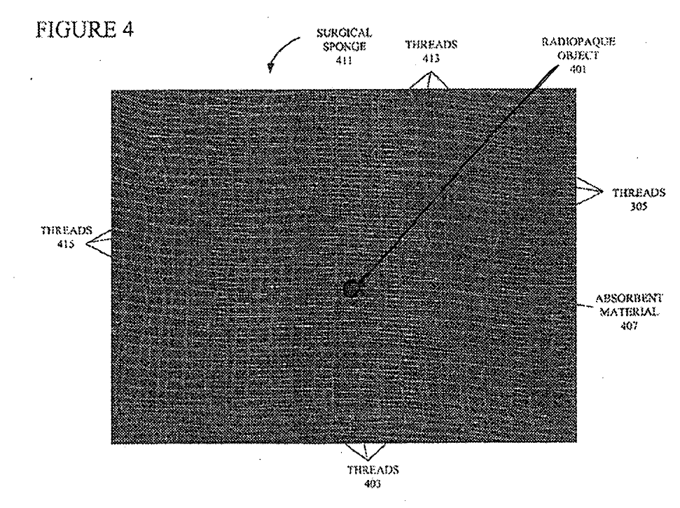

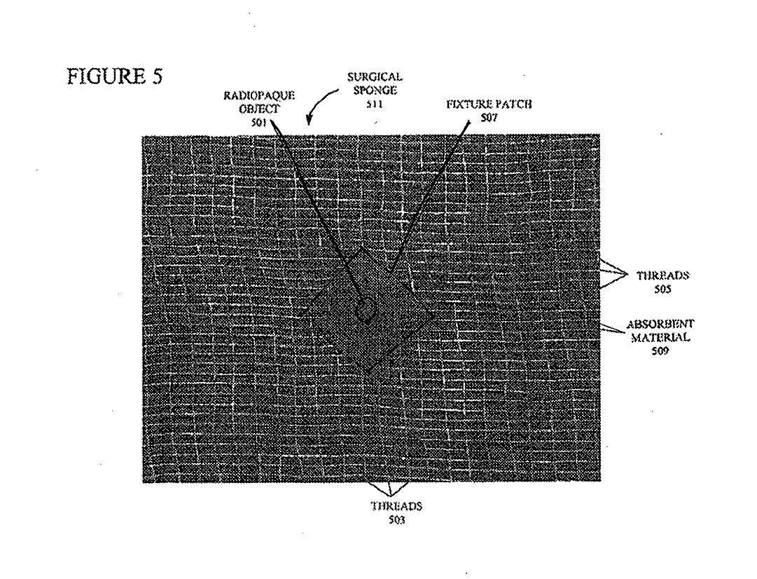

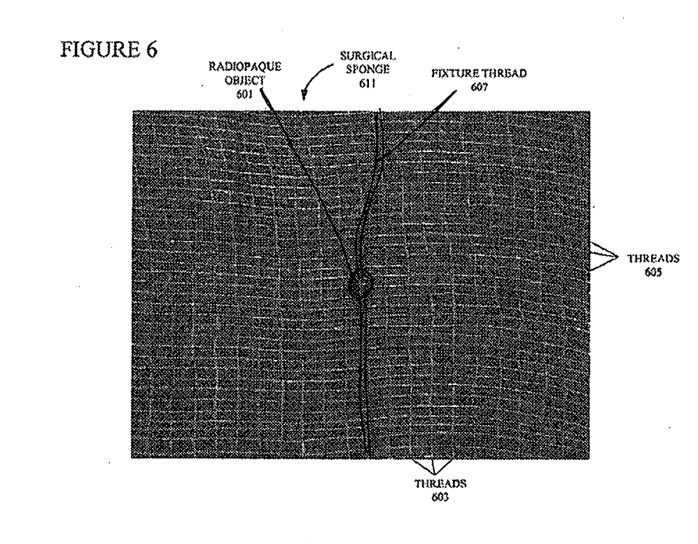

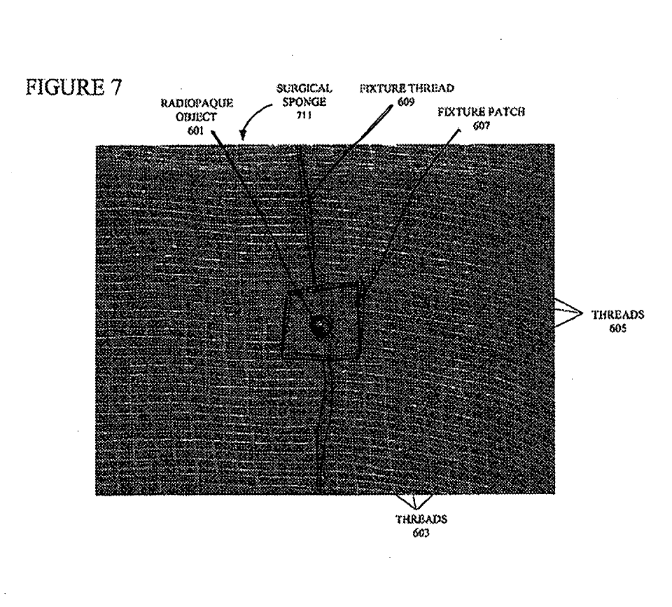

FIGS. 4-7 show alternative methods of affixing a radiopaque object, such as the radiopaque object 301, to a surgical sponge, such as surgical sponges 111 and 311. FIG. 4 is an illustration of an exemplary surgical sponge 411 with a radiopaque object 401 woven or glued into the surgical sponge 411. In other words, the radiopaque object 401 is held between vertical threads 403 and horizontal threads 405 by means of a second layer of vertical threads 413 and a second layer of horizontal threads 415 and/or glued into the surgical sponge 411. FIG. 5 is an illustration of an exemplary surgical sponge 511 with a radiopaque object 501 affixed by means of a fixture patch 507. The fixture patch 507 is a piece of latex, tape or fabric mesh that firmly attaches by means of sewing, gluing or weaving to the radiopaque object 501 and either or both of threads 503 and 505 and absorbent material 509. FIG. 6 is an illustration of an exemplary surgical sponge 611 with a radiopaque object 601 affixed by means of a fixture thread 607. The fixture thread 607 can be either tied to, threaded through or clamped by the radiopaque object 601 and then woven into vertical and horizontal threads 603 and 605. FIG. 7 is an illustration of an exemplary surgical sponge 711 with a radiopaque object 701 affixed by means of both a fixture patch 707, similar to the fixture patch 507 (FIG. 5) and a fixture thread 709, similar to the fixture thread 607 (FIG. 6).

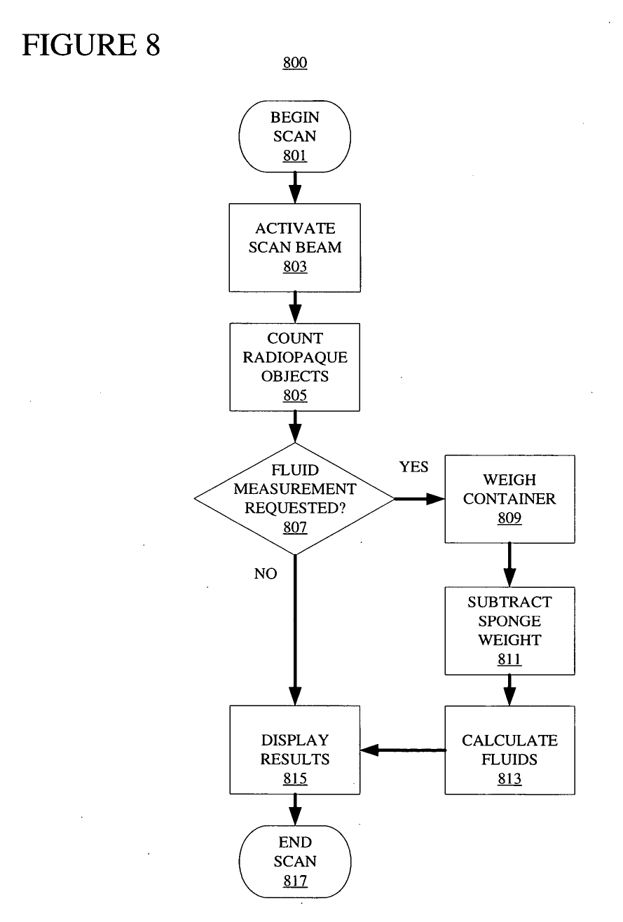

FIG. 8 is a flowchart of a Count Sponge method 800 executed by either the SSTS 100 of FIG. 1 or the SSTS 200 of FIG. 2. The method 800 starts in a Begin Scan step 801 and proceeds immediately to an Activate Scan Beam step 803 in which the radiation source, such as the radiation source 103 (FIG. 1) or the radiation source 203 (FIG. 2) is activated. In the SSTS 100, the radiation source 103 is activated either by the foot pedal 109 or the user controls 113. In the SSTS 200, the radiation source 200 is activated by the computing system 209, either in response to user input on the keyboard 217 or mouse 209 or in response to a timer (not shown) that periodically updates a sponge count produced by the SSTS 200 and displayed on the display 215. In another embodiment of the SSTS 200, the radiation source 203 may be activated in response to the weight sensor in the platform 221 so that information displayed on the display 215 is updated in real time. Control then proceeds to a Count Radiopaque Objects step 805.

In step 805, a sensor detects the number of radiopaque objects such as object 301 (FIG. 3) in the surgical sponges such as surgical sponge 111 in the container 201 by detecting the scanning beam generated by either radiation source 103 or 203. A signal from the sensor is transmitted to the logic (SSTS 100) or the computing system 209 via the connection 207 (SSTS 200), enabling the logic or computing system 209 to calculate the specific number of sponges in the container 101 or 201, respectively. In one embodiment of the invention, surgical sponges of differing configurations (e.g., sizes or types) each contain a radiopaque object of a configuration (e.g., size or shape) that corresponds to the different configuration sponges. Using the different configurations (e.g., sizes or shapes), the logic or computing system 209 processes the signal from the sensor to determine not only a count, but also a specific count for each of the different configuration (e.g., sizes or types) of sponges.

Following step 805, method 800 proceeds to a Fluid Measurement Requested step 807 in which, using the SSTS 200 as an example, the SSTS 200 determines whether information on the collective weight of the sponges in the container 201 is requested. If a weight measurement is not requested, then control proceeds to a Display Results step 815, in which the specific number of sponges calculated in step 805 is displayed on the display 215. In an alternative embodiment, rather than using the display 215, the number may simply be rendered in a display device such as a light emitting diode (LED) device on the platform 221 itself. Of course, if the SSTS 200 does not include a weight sensor in the platform 221, control proceeds directly from step 805 to step 815. If in step 807, method 800 determines that a fluid measurement step is required or requested, then control proceeds to a Weigh Container step 709, in which a weight sensor in the platform sends a signal representing the weight of the container 201 and its contents via the connection 207 to the computing system 209. Control then proceeds to a Subtract Sponge Weight step 811 in which the computing system 209 employs the weight signal, in conjunction with the count signal, to calculate a tare weight for the container 201 and its contents. Control then proceeds to a Calculate Fluids step 813 in which the computing system 209 determines, based upon the tare and the weight signal from the platform 201, the amount of fluids that have been absorbed by the sponges in the container 201. Control then proceeds to the Display Results step 815 in which both the sponge count and the fluid weight is displayed on the display 215 or other display device, such as the display 107 in the case of the SSTS 100. Following step 815, control proceeds to an End Scan step 817 in which processing is complete. Of course, as explained above, method 800 may execute periodically or be initiated by a user.

It is disclosed herein that a surgical supply tracking system (SSTS) in accordance with the disclosed subject matter (e.g., the SSTS 100 depicted in FIG. 1 and/or the SSTS 200 depicted in FIG. 2) is advantageously configurable for enabling system-assisted counting and, optionally, system-performed counting of surgical sponges. One utility of such a SSTS is implementing system-assisted counting of displayed radiopaque objects for allowing operating room personnel to count used surgical sponges through assistance of the SSTS. Another utility of such a SSTS is verification of a system-implemented count of radiopaque objects.

In one embodiment of such a SSTS, the SSTS includes means for visually displaying detected radiopaque objects, means for manually confirming detection of displayed radiopaque objects and means for determining a number of confirmed radiopaque objects. A display (e.g., the display 107 depicted in FIG. 1 or the display 215 depicted in FIG. 2) is an example of the means for displaying detected radiopaque objects. A touchscreen-based response arrangement (e.g., a touchscreen panel overlying the display) and a cursor-based response arrangement (e.g., a screen coordinate selection via a user input device such as a mouse) are examples of the means for manually confirming detection of displayed radiopaque objects. Hardware and/or software logic (e.g., the hardware and/or software logic discussed in reference to FIG. 1) is an example of the means for determining a number of confirmed radiopaque objects. Such hardware and/or software logic are configured for carrying out respective portions of processes, methods and operations in accordance with the inventive disclosures made herein.

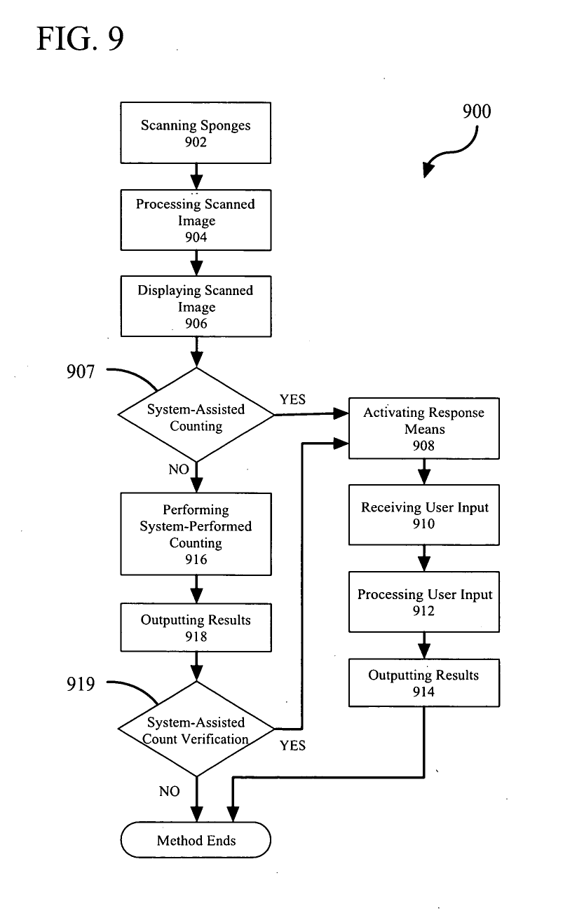

FIG. 9 depicts an embodiment of a method 900 configured for enabling system-assisted counting and, optionally, system-performed counting of surgical sponges. Counting functionality is dependent upon each surgical sponge having attached thereto one or more radiopaque objects in accordance with the inventive disclosures made herein (i.e., radiopague objects that produce a predictable image when scanned). Such surgical sponges are sometimes referred to herein as detectable surgical sponges in reference to the method 900. The SSTS discussed above as being configured for enabling system-assisted counting and, optionally, system-performed counting of surgical sponges is an example of an SSTS capable of carrying out the method 900.

An operation 902 is performed for simultaneously scanning a collection of detectable surgical sponges (e.g., sponges deposited in a sponge container of the SSTS). Scanning is performed with a beam or wave of energy that is obscured or blocked by the one or more radiopaque objects to a different degree than is material from which the surgical sponges are constructed. In this manner, imaging of the radiopaque objects is made possible. In one embodiment, scanning is preferably with an x-ray scanning beam. In other embodiments, scanning is performed with other types of penetrating waves or particles (e.g., such as neutron beams, positron beams, gamma rays, infrared, near-infrared, laser, electromagnetic waves or radio waves). The specific type of scanning beam is not critical to the spirit of the inventions other than that the detectors in the platform must be able to detect the scanning beam with sufficient resolution to enable identification of imaged radiopaque objects by the SSTS and/or a human.

After scanning the collection of surgical sponges, an operation 904 is performed for processing a scanned image, followed by an operation 906 for displaying the scanned image. Processing of the scanned image includes producing a displayable image of the detectable surgical sponges, which may include automated image enhancement for enabling more ready identification of the radiopaque objects within the image. Examples of such image enhancement include, but are not limited to, adjusting contrast, adjusting brightness, and adding color to an otherwise black and white image.

The options of performing system-assisted counting and performing system-performed counting of radiopaque objects are presented at decision block 907. In response to system-assisted counting being selected, an operation 908 is performed for activating a response means (e.g., screen coordinate based response arrangement) that is configured for enabling a user to count the radiopaque objects by selecting radiopaque objects in the displayed scanned image. With the response means activated, an operation 910 is performed for receiving user input that designates imaged radiopaque objects, followed by an operation 912 being performed for processing the user input. Examples of processing the user input include, but are not limited to, summing user inputs to generate a count, confirming user inputs, deactivating selectability of a selected radiopaque object, highlighting a selected radiopaque object, assigning a count number to a selected radiopaque object and/or displaying the count number. Once all user input has been received and processed (e.g., as confirmed by user), an operation 914 is performed for outputting results. Examples of outputting the results of system-assisted counting include, but are not limited to, displaying a total count number, audibly outputting the total count number, outputting a visual representation, (e.g., a picture) of the scanned radiopaque objects and/or printing a report including the total count number. The operations of activating the response means, receiving user input, processing user input and outputting the results represent system-assisted counting functionality in accordance with the inventive disclosures made herein.

Optionally, at the decision block 907, system-performed counting is implemented rather than system-assisted counting. Accordingly, an operation 916 is carried out for performing system-performed counting. In performing system-performed counting, the SSTS determines the number of imaged radiopaque objects without manual selection of the imaged radiopaque objects by a user. Embodiments of system-performed counting are discussed in greater detail above in reference to FIGS. 1, 2 and 8.

After performing the system-performed counting, an operation 918 is performed for outputting results of the system-performed counting. Examples of outputting the results of the system-performed counting include, but are not limited to displaying a total count number, audibly outputting the total count number, and/or printing a report including the total count number.

The option of performing system-assisted verification is presented at decision block 919. In response to system-assisted count verification being selected, the method 900 proceeds with performing system-assisted counting functionality. Accordingly, it will be understood that system-assisted counting verification is a sub-function of system-assisted counting. In performing system-assisted counting verification, the operation 908 is performed for activating the response means and the operation 910 is performed for receiving user input. Examples of processing the user input generally include, but are not limited to, summing inputs to generate a count, confirming user inputs, deactivating selectability of a selected radiopaque object, highlighting a selected radiopaque object, assigning a count number to a selected radiopaque object and/or displaying the count number. Specific to performing system-assisted counting verification, examples of processing the user input include, but are not limited to, comparing a system-generated count of the radiopaque objects with a system-assisted count of the radiopaque objects. Once all user input has been received and processed (e.g., as confirmed by user), the operation 914 is performed for outputting results. Examples of outputting the results of the system-assisted count verification include, but are not limited to, printing a visual representation of the scanned radiopaque objects, outputting count numbers and outputting acknowledgement that the system-performed count has been successfully or unsuccessfully verified.

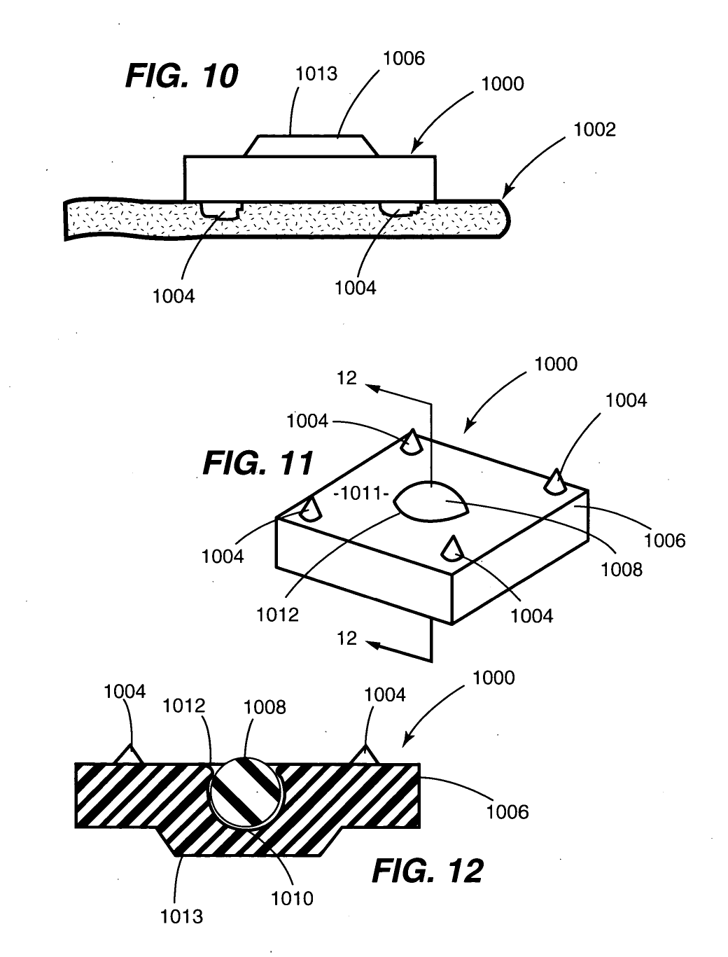

FIGS. 10-23 depict various embodiments of detectable object assemblies. The usefulness of such detectable object assemblies is that they permit small, discrete radiopaque objects to be reliably, efficiently and consistently attached to material configured for absorbing fluids within a body and/or packing bodily structures. Such material may be of any number of configurations. Examples of such material configurations include single or multiple layers of material comprised by woven material, non-woven material, foam material and the like. In one example, such material is provided in the form of a surgical sponge.

In the case of a surgical sponge, these detectable object assemblies may be attached to such material from which the sponge is made during any number of operations in the sponge manufacturing process (i.e., a detectable surgical article manufacturing process). Examples of such operations include, but are not limited to, material unwind operation, material folding operation (i.e., for producing multiple layers of material from a single layer input material), material stacking operation (i.e., for producing multiple layers of material from a single layer input material), material sewing operation, material cutting operation, sponge inspection operation (e.g., where scanning of the radiopaque may be performed in conjunction with or after attachment of the radiopaque object) and sponge packaging operation. Preferably, but not necessarily, the detectable object assemblies are attached during an operation where the material is stationary (e.g., stopped for performing the operation) rather than moving.

As will be appreciated in view of the embodiments of detectable object assemblies presented herein, the detectable object assemblies may be provided at a particular point of attachment in an article manufacturing process and may be provided in any number of different formats. Examples of such formats include, but are not limited to, a roll of attached assemblies, a magazine of discrete assemblies, a magazine of attached assemblies, a magazine of continuous stock (e.g., extruded stock) from which individual assemblies are segmented and the like. The specific format for a given article manufacturing process will be at least partially dependent on specific requirements of that process and/or finished article.

FIGS. 10-12 depict an embodiment of a detectable object structure 1000 attached to a surgical article 1002 by means of a plurality of spaced-apart engagement members 1004. In combination, the detectable object structure 1000 and the surgical article 1002 form a detectable surgical article. The detectable object structure 1000 includes a body 1006 (i.e., an object attachment structure) and a radiopaque object 1008 (i.e., a detectable object). Injection molding is one approach for forming the body 1006.

In accordance with the inventive disclosures made herein, the radiopaque object 1008 is configured for producing predictable profiles when scanned while orientated in different positions. As depicted in FIG. 12, a spherical object made from a radiopaque material (e.g., a steel ball) is one example of the radiopaque object 1008. A volume of radiopaque composition deposited into (e.g., injected into) the cavity 1010 (e.g., a flowable radiopaque composition comprising barium sulfate) is another example of the radiopaque object 1008. The body 1006 is less radiopaque (i.e., more radiographically transparent) than the radiopaque object 1008.

The radiopaque object 1008 is fixedly positioned within a cavity 1010 (i.e., an object-receiving portion) of the body 1006. The body 1006 includes a lip 1012 that overhangs at least a portion of the cavity 1010. When the radiopaque object 1008 and the lip 1012 are suitably sized, the lip 1012 is enables the radiopaque object 1008 to be forcibly inserted into the cavity 1010 and precludes the radiopaque object 1008 from unintentionally separating from the body 1006.

As depicted in FIGS. 11 and 12, the cavity 1010 is accessible through a fabric engagement surface 1011 of the body 1006. Alternately, the cavity 1010 may be accessible through a different surface. For example, in one alternate embodiment (not specifically shown), the cavity 1010 is accessible through a surface opposite the fabric engagement surface 1011 (e.g., the surface 1013).

The body 1006 comprises the plurality of engagement members 1004. Preferably, but not necessarily, the engagement members 1004 are configured specifically for being melted into engagement with material (e.g., fabric) from which the surgical article 1002 is made. Examples of known techniques for melting the engagement members 1004 into engagement with the material include, but are not limited to, thermal heating means, laser heating means and ultrasonic heating means. Alternate means of facilitating engagement of the engagement members 1004 with the material include mechanical deformation of the engagement members 1004, use of a bonding material (e.g., a glue) to chemically facilitate bonding of the engagement members 1004 and use of a solvent to chemically melt the engagement members 1004 into engagement with the fabric.

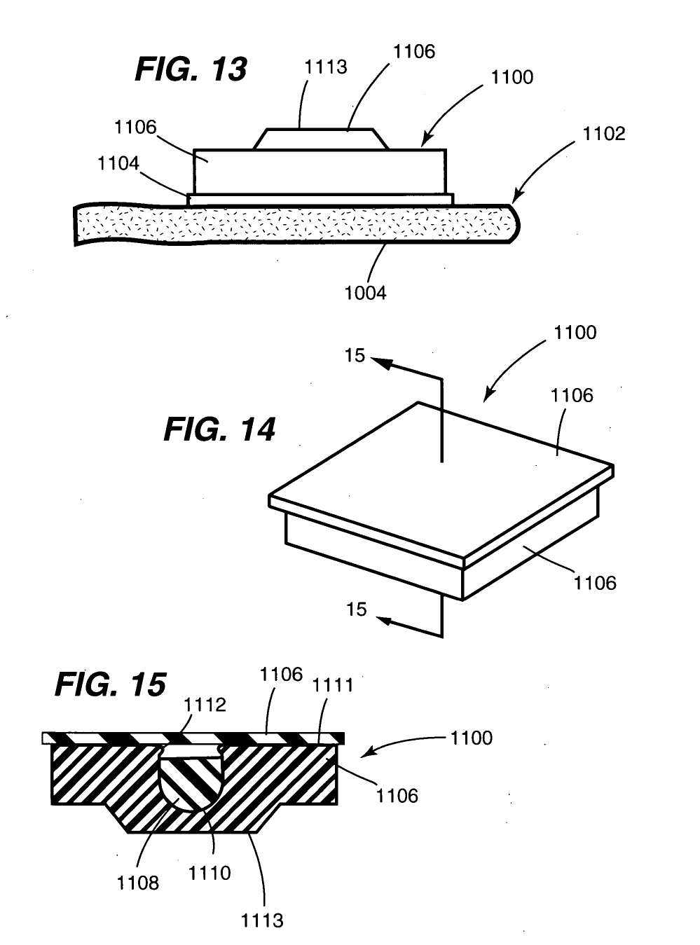

FIGS. 13-15 depict an embodiment of a detectable object structure 1100 attached to a surgical article 1102 by means of a bonding element 1104. In combination, the detectable object structure 1100 and the surgical article 1102 form a detectable surgical article. The detectable object structure 1100 includes a body 1106 (i.e., an object attachment structure) and a radiopaque object 1108 (i.e., a detectable object). Injection molding is a preferred approach for forming the body 1106.

In accordance with the inventive disclosures made herein, the radiopaque object 1108 is configured for producing predictable profiles when scanned while orientated in different positions. As depicted in FIG. 15, a volume of radiopaque composition deposited into (e.g., injected into) the cavity 1110 (e.g., a flowable radiopaque composition comprising barium sulfate) is an example of the radiopaque object 1108. A spherical object made from a radiopaque material (e.g., a steel ball) is another example of the radiopaque object 1108. The body 1106 is less radiopaque (i.e., more radiographically transparent) than the radiopaque object 1108.

The radiopaque object 1108 is fixedly positioned within a cavity 1110 (i.e., an object-receiving portion) of the body 1106. The body 1106 may include includes a lip 1112 that overhangs at least a portion of the cavity 1110. When the radiopaque object 1108 and lip 1112 are suitably sized, the lip 1112 precludes the radiopaque object 1108 from unintentionally separating from the body 1106.

As depicted in FIG. 15, the cavity 1110 is accessible through a fabric engagement surface 1111 of the body 1106. Alternately, the cavity 1110 may be accessible through a different surface. For example, in one alternate embodiment (not specifically shown), the cavity 1110 is accessible through a surface opposite the fabric engagement surface 1111 (e.g., the surface 1113).

The bonding element 1104 is attached to body 1106. Examples of the bonding element include, but are not limited to, a layer of hot melt adhesive, a layer of pressure-sensitive adhesive and a layer of solvent-activatable adhesive. Preferably, but not necessarily, the bonding element 1104 is an integral component of the detectable object structure 1100 (i.e., a pre-fabricated assembly). Examples of known techniques for securing the bonding element 1104 to the body and/or into engagement with the material from which the surgical sponge is made include, but are not limited to, thermal heating means, laser heating means, ultrasonic heating means, pressure application means, mechanical deformation means, and/or solvent application means.

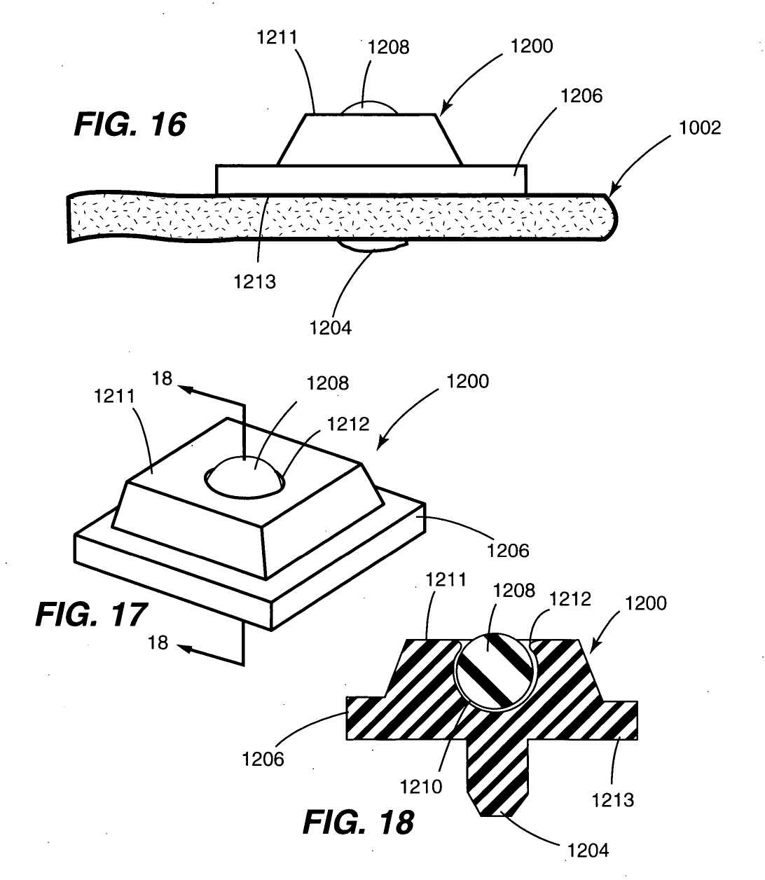

FIGS. 16-18 depict an embodiment of a detectable object structure 1200 attached to a surgical article 1202 by means of an engagement member 1204. In combination, the detectable object structure 1200 and the surgical article 1202 form a detectable surgical article. The detectable object structure 1200 includes a body 1206 (i.e., an object attachment structure) and a radiopaque object 1208 (i.e., a detectable object). Injection molding is a preferred approach for forming the body 1206.

In accordance with the inventive disclosures made herein, the radiopaque object 1208 is configured for producing predictable profiles when scanned while orientated in different positions. As depicted in FIG. 18, a spherical object made from a radiopaque material (e.g., a steel ball) is one example of the radiopaque object 1208. A volume of radiopaque composition deposited into (e.g., injected into) the cavity 1210 (e.g., a flowable radiopaque composition comprising barium sulfate) is another example of the radiopaque object 1208. The body 1206 is less radiopaque (i.e., more radiographically transparent) than the radiopaque object 1208.

The radiopaque object 1208 is fixedly positioned within a cavity 1210 (i.e., an object-receiving portion) of the body 1206. The body 1206 includes a lip 1212 that overhangs at least a portion of the cavity 1210. When the radiopaque object 1208 and the lip 1212 are suitably sized, the lip 1212 is enables the radiopaque object 1208 to be forcibly inserted into the cavity 1210 and precludes the radiopaque object 1208 from unintentionally separating from the body 1206. As depicted in FIGS. 16-18, the cavity 1210 is accessible through a surface 1211 opposite a fabric engagement surface 1213 of the body 1206.

The body 1206 comprises the engagement member 1204. Preferably, but not necessarily, the engagement member 1204 is configured specifically for being melted into engagement with material (e.g., fabric) from which the surgical article 1202 is made. Examples of known techniques for melting the engagement member 1204 into engagement with the material include, but are not limited to, thermal heating means, laser heating means and ultrasonic heating means. Alternate means of facilitating engagement of the engagement members 1204 with the material include mechanical deformation of the engagement member 1204, use of a bonding material (e.g., a glue) to chemically facilitate bonding of the engagement members 1204 and use of a solvent to chemically melt the engagement members 1204 into engagement with the fabric.

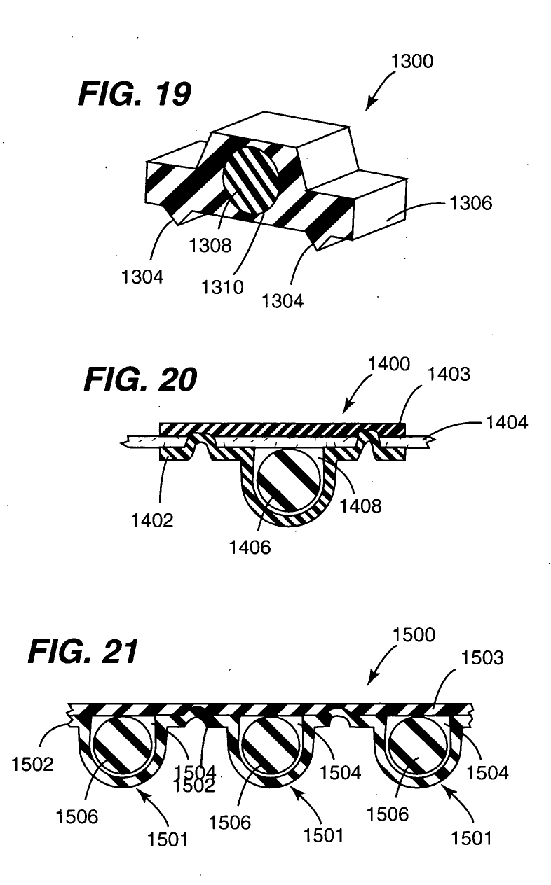

FIG. 19 depicts an embodiment of an extruded detectable object structure 1300 configured for being attached to a surgical article by means of a plurality of spaced-apart engagement members 1304. In combination, the detectable object structure 1300 and the surgical article form a detectable surgical article. The detectable object structure 1300 includes a body 1306 (i.e., an object attachment structure) and a radiopaque object 1308 (i.e., a detectable object).

The body 1306 is formed via an extrusion process. Preferably, but not necessarily, the radiopaque object 1308 is formed in unison with the body 1306 via what is typically termed a co-extrusion process. In such a process, the body 1306 is extruded simultaneously with the radiopaque object 1308 (e.g., formed around the radiopaque object 1308). A radiopaque composition (e.g., an extrudable composition comprising barium sulfate) co-extruded with the body 1306 is one example of the radiopaque object 1308. A length of wire that has the body 1306 extruded around it is another example of the radiopaque object 1308. Still another example of the radiopaque object 1308 is a volume of radiopaque composition deposited into (e.g., injected into) a cavity 1310 of the body 1306 (e.g., a flowable radiopaque composition) after the body is formed (i.e., extruded and, optionally, cut to final length). In accordance with the inventive disclosures made herein, the radiopaque object 1308 is configured for producing predictable profiles when scanned while orientated in different positions. The body 1306 is less radiopaque (i.e., more radiographically transparent) than the radiopaque object 1308.

The body 1306 comprises the plurality of engagement members 1304. Preferably, but not necessarily, the engagement members 1304 are configured specifically for being melted into engagement with material (e.g., fabric) from which the surgical article 1302 is made. Examples of known techniques for melting the engagement members 1304 into engagement with the material include, but are not limited to, thermal heating means, laser heating means and ultrasonic heating means. Alternate means of facilitating engagement of the engagement members 1304 with the material include mechanical deformation of the engagement members 1304, use of a bonding material (e.g., a glue) to chemically facilitate bonding of the engagement members 1304 and use of a solvent to chemically melt the engagement members 1304 into engagement with the fabric.

FIG. 20 depicts an embodiment of a detectable surgical article 1400 including a pair of engaged bodies (i.e., a first body 1402 and a second 1403) attached to a surgical article 1404 and fixedly engaged with a radiopaque object 1406. The surgical article 1404 is disposed between the pair of engaged bodies (i.e., an object attachment structure). The pair of engaged bodies is attached by means such as, for example, ultrasonic welding, laser welding, mechanical staking and solvent bonding. The first body 1402 includes a cavity 1408 (i.e., an object-receiving portion) having the radiopaque object 1406 disposed therein. In other embodiments (not specifically shown), the cavity 1408 may be substituted with a passage or a channel configured for receiving the radiopaque object 1406. It is disclosed that an operation such as mechanical stacking, ultrasonic welding, laser welding, chemical bonding, solvent welding or the like may be used for securing the radiopaque object in the object-receiving portion.

The engaged bodies may be formed by any number of techniques. Examples of such techniques for pre-forming the engaged bodies include, but are not limited to, injection molding, extrusion, and vacuum forming. It is also disclosed that the engaged bodies may be formed in-situ (i.e., in-line with forming the surgical article) from flexible material such as sheets of a fabric material or polymeric material (i.e., the object-receiving portion is a pocket of an envelope/pouch).

In accordance with the inventive disclosures made herein, the radiopaque object 1406 is configured for producing predictable profiles when scanned while orientated in different positions. As depicted in FIG. 20, a spherical object made from a radiopaque material (e.g., a steel ball) is one example of the radiopaque object 1406. A volume of radiopaque composition deposited into (e.g., injected into) the cavity 1408 (e.g., a flowable radiopaque composition comprising barium sulfate) is another example of the radiopaque object 1406. The pair of engaged bodies is less radiopaque (i.e., more radiographically transparent) than the radiopaque objects 1408.

In another embodiment (not specifically shown), the pair of engaged bodies depicted in FIG. 20 may be attached to each other in a clamshell fashion. Accordingly, a delectable object structure comprising a body having such a clamshell configuration is preferably attached to an edge portion of a surgical article.

FIG. 21 depicts an embodiment of a segment 1500 of continuously formed detectable object assemblies 1501. The segment 1500 includes a pair of engaged bodies (i.e., a first body 1502 and a second body 1503) attached in a manner that defines object-receiving portions 1504. Each one of the object-receiving portions 1504 includes a radiopaque object 1506 disposed therein.

As depicted in FIG. 21, each one of the object-receiving portions 1504 is a cavity. In other embodiments (not specifically shown), each one of the object-receiving portions 1504 may be a passage or a channel configured for receiving the radiopaque object. It is disclosed that an operation such as mechanical stacking, ultrasonic welding, laser welding, chemical bonding, solvent welding or the like may be used for securing the radiopaque object in the object-receiving portion.

As depicted in FIG. 21, each object-receiving portion 1504 is formed in only the first body 1502. In other embodiments (not specifically shown), the first body 1502 and the second body 1503 jointly define each object-receiving portion 1502 (e.g., a pocket of an envelope/pouch).

The engaged bodies may be formed by any number of techniques. Examples of such techniques for pre-forming the engaged bodies include, but are not limited to, injection molding, extrusion, and vacuum forming. It is also disclosed that the engaged bodies may be formed in-situ from flexible material such as sheets of a fabric material or polymeric material (i.e., the object-receiving portion 1504 is a pocket of an envelope/pouch).

In accordance with the inventive disclosures made herein, the radiopaque object 1506 is configured for producing predictable profiles when scanned while orientated in different positions. As depicted in FIG. 21, a spherical object made from a radiopaque material (e.g., steel) is one example of the radiopaque object 1506. A volume of radiopaque composition deposited into (e.g., injected into) each cavity 1504 (e.g., a flowable radiopaque composition comprising barium sulfate) is another example of the radiopaque object 1506. The pair of engaged bodies is less radiopaque (i.e., more radiographically transparent) than the radiopaque objects 1506.

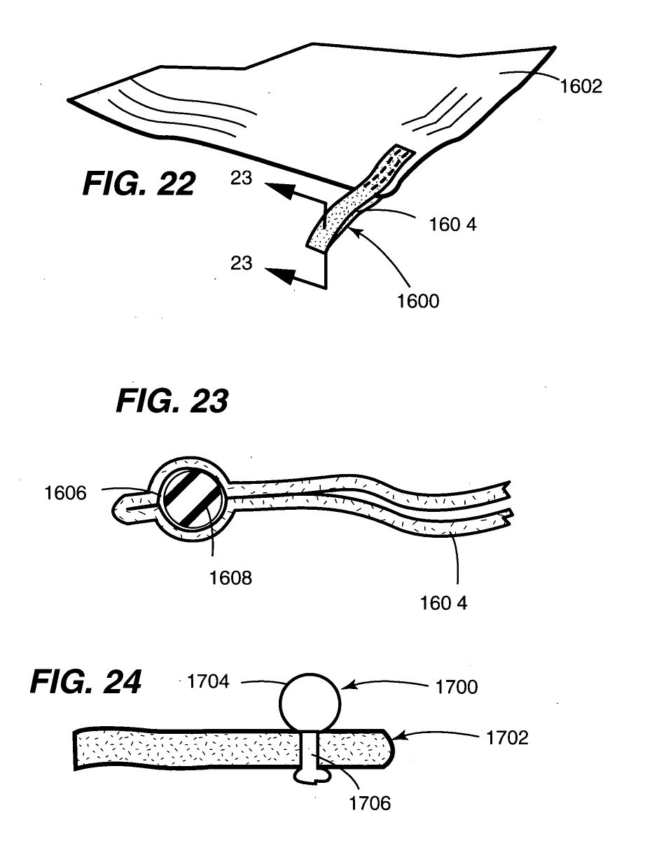

FIGS. 22 and 23 depict an embodiment of a flexible detectable object structure 1600 attached to a surgical article 1602. In combination, the detectable object structure 1600 and the surgical article 1202 form a detectable surgical article. The flexible detectable object structure 1600 includes a strip of flexible material 1604 (e.g., fabric or flexible polymeric film) defining a pocket 1606 (i.e., an object-receiving portion) having a radiopaque object 1608 disposed therein. In other embodiments (not specifically shown), the pocket 1606 is defined by a plurality of separate strips of flexible material. The pocket 1604 may be formed by any number of techniques. Examples of such techniques include, but are not limited to, sewing, bonding, mechanical staking, laser welding, ultrasonic welding, chemical bonding, and solvent welding. In accordance with the inventive disclosures made herein, the radiopaque object 1608 is configured for producing predictable profiles when scanned while orientated in different positions. As depicted in FIG. 23, a spherical object made from a radiopaque material (e.g., a steel ball) is one example of the radiopaque object 1608.

FIG. 24 depicts an embodiment of a one-piece detectable object structure 1700 attached to a surgical article 1702. The one-piece detectable object structure 1700 includes a main portion 1704 (i.e., a radiopaque object) and an engagement member 1706 (i.e., an object attachment structure). The attachment member 1706 is configured for securing the detectable object structure 1700 to the surgical article 1702. Preferably, but not necessarily, the engagement member 1706 is configured specifically for being melted into engagement with material (e.g., fabric) from which the surgical article 1702 is made. Examples of known techniques for melting the engagement member 1706 into engagement with the material include, but are not limited to, thermal heating means, laser heating means and ultrasonic heating means. Alternate means of facilitating engagement of the engagement member 1706 with the material include mechanical deformation of the engagement member 1706, use of a bonding material (e.g., a glue) to chemically facilitate bonding of the engagement member 1706 and use of a solvent to chemically melt the engagement member 1706 into engagement with the fabric.

In accordance with the inventive disclosures made herein, at least the main portion 1704 of the detectable object structure 1700 is configured for producing predictable profiles when scanned while orientated in different positions. As depicted in FIG. 24, one example of the main portion 1704 is a generally spherically shaped object and one example of the attachment member is a slender rod attached to the main portion 1704. Examples of radiopaque materials from which the detectable object structure 1700 may be made include, but are not limited to, metal (e.g., steel) and a formable radiopaque composition (e.g., a moldable radiopaque composition comprising barium sulfate).

In another embodiment (not specifically shown), a detectable object structure includes a radiopaque object configured for producing predictable profiles when scanned while orientated in different positions and a bondable coating at least partially covering the radiopaque object. For example, a round steel ball is encapsulated with a meltable polymeric material. Such a detectable object structure may be applied to surgical sponge material by application of, for example, heat, a suitable solvent or a suitable adhesive.

While various embodiments of the application have been described, it will be apparent to those of ordinary skill in the art that many more embodiments and implementations are possible that are within the scope of this invention. Accordingly, the invention is not to be restricted except in light of the attached claims and their equivalents.

1. A detectable surgical article, comprising:
material configured for at least one of absorbing fluids within a body and packing bodily structures;
a radiopaque object configured for producing predictable profiles when scanned while orientated in different positions; and
means configured for attaching the radiopaque object to said material, wherein said means is attached to said material and wherein the radiopaque object is fixedly engaged by said means.

2. The detectable surgical article of claim 1 wherein said means includes an object-receiving portion having the radiopaque object at least partially disposed therein.

3. The detectable surgical article of claim 2 wherein:
said means includes at least one strip of flexible material; and
the object-receiving portion includes a pocket formed by said at least one strip of flexible material.

4. The detectable surgical article of claim 2 wherein:
said means includes a pre-formed body; and
the object-receiving portion includes at least one of a channel, a passage and a cavity formed in a surface of the pre-formed body.

5. The detectable surgical article of claim 4 wherein said means includes an object retaining material disposed within said at least one of the channel, the passage and the cavity for limiting movement of the radiopaque object relative to said means.

6. The detectable surgical article of claim 2 wherein:
said means includes a pair of engaged pre-formed bodies; and
the object-receiving portion includes at least one of a channel, a passage and a cavity formed defined by at least one of said engaged pre-formed bodies.

7. The detectable surgical article of claim 1 wherein:
said means includes a fabric engaging surface; and
said means includes a bonding material disposed between the fabric engaging surface and said material.

8. The detectable surgical article of claim 1 wherein:
said means includes a fabric engaging surface; and
said means includes a material engagement member extending from the fabric engaging surface and being engaged with said material for limiting movement of the transponder attachment structure relative to said material.

9. A detectable surgical article, comprising:
material configured for at least one of absorbing fluids within a body and packing bodily structures;
an object attachment structure attached to said material; and
a radiopaque object configured for producing predictable profiles when scanned while orientated in different positions, wherein the radiopaque object is fixedly engaged by the object attachment structure.

10. The detectable surgical article of claim 9 wherein the object attachment structure includes an object-receiving portion having the radiopaque object at least partially disposed therein.

11. The detectable surgical article of claim 10 wherein:
the object attachment structure includes at least one strip of flexible material; and
the object-receiving portion includes a pocket formed by said at least one strip of flexible material.

12. The detectable surgical article of claim 10 wherein:
the object attachment structure includes a pre-formed body; and
the object-receiving portion includes at least one of a channel, a passage and a cavity formed in a surface of the pre-formed body.

13. The detectable surgical article of claim 12 wherein the object attachment structure includes an object retaining material disposed within said at least one of the channel, the passage and the cavity for limiting movement of the radiopaque object relative to the object attachment structure.

14. The detectable surgical article of claim 10 wherein:
the object attachment structure includes a pair of engaged pre-formed bodies; and
the object-receiving portion includes at least one of a channel, a passage and a cavity formed defined by at least one of said engaged pre-formed bodies.

15. The detectable surgical article of claim 9 wherein:
the object attachment structure includes a fabric engaging surface; and
the object attachment structure includes a bonding material disposed between the fabric engaging surface and said material.

16. The detectable surgical article of claim 9 wherein:
the object attachment structure includes a fabric engaging surface; and
the object attachment structure includes a material engagement member extending from the fabric engaging surface and being engaged with said material for limiting movement of the transponder attachment structure relative to said material.

17. A detectable object structure, comprising:
a radiopaque object configured for producing predictable profiles when scanned while orientated in different positions; and
an object attachment structure including an object-receiving portion having the radiopaque object at least partially disposed therein.

18. The detectable object structure of claim 17 wherein:
the object attachment structure includes at least one strip of flexible material; and
the object-receiving portion includes a pocket formed by said at least one strip of flexible material.

19. The detectable object structure of claim 17 wherein:
the object attachment structure includes a pre-formed body; and
the object-receiving portion includes at least one of a channel, a passage and a cavity formed in a surface of the pre-formed body.

20. The detectable object structure of claim 19 wherein the object attachment structure includes an object retaining material disposed within said at least one of the channel, the passage and the cavity for securing the radiopaque object in place.

21. The detectable object structure of claim 17 wherein:
The object attachment structure includes a pair of engaged pre-formed bodies; and
the object-receiving portion includes at least one of a channel, a passage and a cavity formed defined by at least one of said engaged pre-formed bodies.

22. The detectable object structure of claim 17 wherein:
the object attachment structure includes a fabric engaging surface; and
the object attachment structure includes a bonding material disposed between the fabric engaging surface and said material.

23. The detectable object structure of claim 17 wherein:
the object attachment structure includes a fabric engaging surface; and
the object attachment structure includes a material engagement member extending from the fabric engaging surface and being engaged with said material for securing the transponder attachment structure to said material.
